(19) United States
(12) Patent Application Publication
(10) Pub. No.: US 2024/0363322 A1
(43) Pub. Date: Oct. 31, 2024

(54) NEBULISER OUTLET ASSEMBLY

(71) Applicant: Micromass UK Limited, Wilmslow (GB)

(72) Inventors: Suloke Mathai, Macclesfield (GB); Ian David Trivett, Cheadle (GB)

(73) Assignee: Micromass UK Limited, Wilmslow (GB)

(21) Appl. No.: 18/686,595

(22) PCT Filed: Aug. 31, 2022
(86) PCT No.: PCT/GB2022/052229

§ 371 Date: Feb. 26, 2024

(30) Foreign Application Priority Data
Aug. 31, 2021 (GB) 2112402.9

(51) Int. Cl. H01J 49/04; G01N 27/623; H01J 49/16

(52) U.S. Cl. CPC H01J 49/045; H01J 49/167; G01N 27/623

(57) ABSTRACT

A nebuliser outlet assembly comprises an inlet end and an outlet end, and a first channel (24) arranged between the inlet end and the outlet end, wherein the first channel is configured to receive a capillary. The nebuliser outlet assembly comprises a first part (40) and one or more second parts (50), wherein the one or more second parts are removably attachable to the first part. The nebuliser outlet assembly is configured such that the first channel is formed from the first part and the one or more second parts when the one or more second parts are attached to the first part. The first part comprises an open-sided channel that corresponds to the first channel for at least some of its length. The nebuliser outlet assembly is configured such that when the one or more second parts are removed from the first part, said open-sided channel is exposed.

### CROSS-REFERENCE TO RELATED APPLICATION

This application claims priority from and the benefit of United Kingdom patent application No. 2112402.9 filed on 31 Aug. 2021. The entire contents of this application are incorporated herein by reference.

### FIELD OF INVENTION

The present invention relates generally to ion sources and in particular to nebulisers for ion sources.

### BACKGROUND

Ionisation techniques such as Electrospray Ionisation (ESI) utilise a nebuliser to generate a spray of droplets.

Such nebulisers typically comprise a liquid capillary and a gas capillary. The liquid capillary is typically arranged coaxially within the gas capillary, with the liquid-emitting outlet of the liquid capillary extending beyond the distal end of the gas capillary. A flow of liquid supplied to the liquid capillary is nebulised by a nebulising gas flow supplied to the gas capillary.

The Applicant believes there remains scope for improvements to nebulisers.

### SUMMARY

According to an aspect, there is provided a nebuliser outlet assembly comprising:
an inlet end and an outlet end; and
a first channel arranged between the inlet end and the outlet end, wherein the first channel is configured to receive a capillary;
wherein the nebuliser outlet assembly comprises a first part and one or more second parts, wherein the one or more second parts are removably attachable to the first part;
wherein the nebuliser outlet assembly is configured such that the first channel is formed from the first part and the one or more second parts when the one or more second parts are attached to the first part; and
wherein the nebuliser outlet assembly is configured such that when the one or more second parts are removed from the first part, at least some of the first channel is exposed.

Various embodiments are directed to a nebuliser outlet assembly (a nebuliser outlet tip) having an inlet end and an outlet end. A first channel is arranged between the inlet end and the outlet end. One or more second channels may also be arranged between the inlet end and the outlet end. The nebuliser outlet assembly may be configured such that, when a capillary is received by the first channel (and when the one or more second parts are attached to the first part), gas received by the nebuliser outlet assembly will pass to the outlet end via the one or more second channels, and will nebulise liquid emitted from the capillary.

The nebuliser outlet assembly is formed from (at least) a first part and one or more second parts, where the one or more second parts are removably attachable to the first part. The nebuliser outlet assembly is configured such that when the one or more second parts are attached to the first part (i.e. when the nebuliser outlet assembly is assembled), the first channel (and optionally the one or more second channels) is formed from the first part and from the one or more second parts. This may be such that when assembled, i.e. when the one or more second parts are attached to the first part, most or all of the first channel and optionally most or all of one or more second channels (e.g. except its inlet(s) and/or outlet(s)) is substantially concealed, e.g. from a user of the nebuliser outlet assembly.

The nebuliser outlet assembly is also configured such that when the one or more second parts are removed from the first part, i.e. when the nebuliser outlet assembly is disassembled, at least some of the first channel and optionally the one or more second channels is exposed, e.g. to a user of the nebuliser outlet assembly.

As will be described in more detail below, this multi-part construction provides a number of benefits. In particular, this configuration means that, during assembly, a user may install a liquid capillary into the (first channel of the) nebuliser outlet assembly when the one or more second parts have been removed from the first part. This allows the user e.g. to (visually) verify and/or measure whether the liquid capillary has been properly installed into the (first channel of the) nebuliser outlet assembly.

This then means that spatial parameters of the nebuliser outlet assembly, in particular those parameters which affect the interaction between the liquid emitted by the capillary and the nebulising gas, can be precisely controlled and/or substantially fixed. Thus, variation in the performance of the nebuliser can be significantly reduced, for example, from one use to the next (for example after uninstalling and re-installing a liquid capillary), and from one nebuliser to the next.

Furthermore, embodiments facilitate cleaning of the internal features (i.e. the first and/or one or more second channels) of the nebuliser outlet assembly, e.g. by providing access to the internal features (i.e. the first and/or one or more second channels) of the nebuliser outlet assembly, e.g. when the one or more second parts are removed from the first part.

Various embodiments accordingly provide an improved nebuliser outlet assembly.
wherein the nebuliser outlet assembly is configured such that when the one or more second parts are attached to the first part, the first channel and/or one or more second channels are concealed; and
wherein the nebuliser outlet assembly is configured such that when the one or more second parts are removed from the first part, the first channel and/or one or more second channels are visible.

The nebuliser outlet assembly may comprise one or more gas inlets. One or more or each of the gas inlets may be arranged at the inlet end. Additionally or alternatively, one or more of each of the gas inlets may be arranged elsewhere between the inlet end and the outlet end, such as on a side wall of the nebuliser outlet assembly between the inlet end and the outlet end.

The nebuliser outlet assembly may be configured such that (when assembled and) when a capillary is received by the first channel, gas provided to (one or more gas inlets of) the nebuliser outlet assembly can pass via the one or more second channels to the outlet end.

The nebuliser outlet assembly may be configured such that (when assembled and) when a capillary is received by the first channel, gas provided to (one or more gas inlets of) the nebuliser outlet assembly can pass via the one or more second channels to the outlet end, and can nebulise liquid emitted by the capillary.

The nebuliser outlet assembly may comprise an outlet aperture arranged at the outlet end. The outlet may be formed in (only) one of the first or second parts. Alternatively, the nebuliser outlet assembly may be configured such that the outlet aperture is formed from the first part and the one or more second parts when the one or more second parts are attached to the first part (i.e. when the outlet assembly is assembled).

The assembly may comprise one or more second channels arranged between the inlet end and the outlet end. The one or more second channels may be configured (when the outlet assembly is assembled) to pass gas (from the one or more gas inlets) to or adjacent to the outlet aperture. The nebuliser outlet assembly may be configured such that (when assembled and) when a capillary is received by the first channel, gas provided to (one or more gas inlets of) the nebuliser outlet assembly can pass via the one or more second channels to the outlet end, and can nebulise liquid emitted by the capillary, such that a spray of droplets is emitted from the aperture.

The first channel is arranged between the inlet end and the outlet end. The first channel may extend along the entire (axial) length of the nebuliser outlet assembly between the inlet end and the outlet end. The first channel may have an inlet arranged at the inlet end of the nebuliser outlet assembly, and may have an outlet arranged at the outlet end of the nebuliser outlet assembly.

Each of the one or more second channels may be arranged between the inlet end and the outlet end. One or more or each of the one or more second channels may extend along the entire (axial) length of the nebuliser outlet assembly between the inlet end and the outlet end, and/or one or more or each of the one or more second channels may extend along only part (some but not all) of the length of the nebuliser outlet assembly between the inlet end and the outlet end. One or more or each second channels may have an inlet arranged at the inlet end of the nebuliser outlet assembly or elsewhere between the inlet end and the outlet end, and may have an outlet arranged at or close to the outlet end of the nebuliser outlet assembly.

The first channel may be separate from the one or more second channels at the inlet end. Alternatively, the one or more second channels may diverge from the first channel at one or more points along the length of the nebuliser outlet assembly.

The first channel may be separate from the one or more second channels for at least some (but not all) of the length of the nebuliser outlet assembly.

The first channel and the one or more second channels may converge at a convergence region within the nebuliser outlet assembly. The convergence region may be arranged at or close to the outlet end and/or the outlet aperture.

Alternatively, the nebuliser outlet assembly may be configured such that the first channel and the one or more second channels other than (do not) converge within the nebuliser outlet assembly.

Where there is a convergence region, the nebuliser outlet assembly may be configured such that gas provided to the nebuliser outlet assembly can meet (and nebulise) liquid emitted by the capillary in the convergence region. In other words, the convergence region may be a nebulisation region. In these embodiments, the nebuliser outlet assembly may be configured such that, (when assembled and) when the capillary is installed in the (first channel of the) nebuliser outlet assembly, an outlet of the capillary is arranged in the convergence region.

Alternatively, the nebuliser outlet assembly may be configured such that gas provided to the nebuliser outlet assembly can meet (and nebulise) liquid emitted by the capillary in a nebulisation region downstream of the convergence region. Thus, the nebuliser outlet assembly may be configured such that, (when assembled and) when the capillary is installed in the (first channel of the) nebuliser outlet assembly, the outlet of the capillary is arranged downstream of (protrudes beyond) the convergence region. In these embodiments, the nebulisation region may be within the nebuliser outlet assembly, or external from (downstream of) the nebuliser outlet assembly.

Where the nebuliser outlet assembly is configured such that the first channel and the one or more second channels other than (do not) converge within the nebuliser outlet assembly, the nebuliser outlet assembly may be configured such that (when assembled) gas provided to the nebuliser outlet assembly can meet (and nebulise) liquid emitted by the capillary in a nebulisation region external from (downstream of) the nebuliser outlet assembly.

In various embodiments, the nebuliser outlet assembly may be configured such that, (when assembled and) when the capillary is installed in the (first channel of the) nebuliser outlet assembly, the outlet of the capillary is arranged within the nebuliser outlet assembly, such as being withdrawn from the outlet aperture.

The nebuliser outlet assembly may instead be configured such that, (when assembled and) when the capillary is installed in the (first channel of the) nebuliser outlet assembly, the outlet of the capillary protrudes beyond the outlet aperture. In these embodiments, the nebuliser outlet assembly may be configured such that, (when assembled and) when the liquid capillary is installed in the (first channel of the) nebuliser outlet assembly, gas received by the nebuliser outlet assembly can pass to the outlet aperture via the one or more second channels, and can be emitted from the outlet end such as from the outlet aperture (optionally through an annulus between the capillary and the outlet aperture) so as to nebulise liquid emitted by the outlet of the capillary.

The outlet aperture may have a first area such as a first cross-sectional area. The outlet aperture may have a first diameter.

At least part of the first channel may have a second area such as a second cross-sectional area. At least part of the first channel may have a second diameter.

The second (cross-sectional) area may be approximately equal to or slightly larger than the (cross-sectional) area of the liquid capillary. The second diameter may be approximately equal to or slightly larger than the outer diameter of the liquid capillary.

The second (cross-sectional) area may be less than the first (cross-sectional) area. The second diameter may be less than the first diameter.

However, it would be possible for the second (cross-sectional) area to be equal to or larger than the first (cross-sectional) area (and for the second diameter to be equal to or larger than the first diameter).

The nebuliser outlet assembly may be configured such that (when assembled and) when a capillary is received by the first channel, the capillary is retained relative to (such as centrally to) the outlet aperture by the first channel, for example by the at least part of the first channel that has the second area (that has the second diameter).

In some embodiments, the first channel is coaxially aligned with the outlet aperture. However, in other embodiments, the first channel may be offset from (not coaxially aligned with) the outlet aperture (in a radial direction).

At least part of the first channel may have an area (a cross-sectional area) that is less than an area (a cross-sectional area) of the outlet aperture. This may be so as to retain (the outlet of) a liquid capillary centrally to (in coaxial alignment with) the outlet aperture (when the assembly is assembled and) when the liquid capillary is installed in the nebuliser outlet assembly.

Thus, embodiments provide a nebuliser outlet assembly in which a liquid capillary can be retained in a substantially fixed position relative to (such as centrally to) the outlet aperture while at the same time allowing a nebulising gas to be provided to the outlet end such as to the outlet aperture. Retaining the liquid capillary relative to (such as centrally to) the outlet aperture in this manner can significantly reduce variation in the performance of the nebuliser, for example from one use to the next (for example after uninstalling and re-installing a liquid capillary), and from one nebuliser to the next.

The nebuliser outlet assembly comprises a first part and one or more second parts, where the one or more second parts are removably attachable to the first part. The nebuliser outlet assembly may comprise (and in embodiments does comprise) one or more third parts, which may be configured as desired.

The nebuliser outlet assembly may comprise a single second part which is removably attachable to the first part, or multiple (two or more) second parts which are each removably attachable to the first part.

The one or more second parts may be removably attachable to the first part using any suitable means. For example, the first part and/or the one or more second parts may (each) include a suitable attachment fitting(s) to allow the one or more second parts to be removably attached to the first part. Alternatively, the nebuliser outlet assembly may include one or more (separate) attachment fittings, such as for example one or more clamps, one or more clips, one or more screw fittings, one or more straps, and the like, configured to allow the one or more second parts to be removably attached to the first part.

The nebuliser outlet assembly is configured such that the first channel and/or the one or more second channels are formed from the first part and from the one or more second parts when the one or more second parts are attached to the first part.

Thus, the nebuliser outlet assembly may be configured such that when the first part and the one or more second parts are assembled together (i.e. when the nebuliser outlet assembly is assembled), the first channel and/or the one or more second channels (i.e. as described above) are formed from the first part and from the one or more second parts. This may such that when assembled, most or all of the first channel and/or one or more second channels (e.g. except their inlet(s) and/or outlet(s)) are substantially concealed, e.g. from a user of the nebuliser outlet assembly.

The nebuliser outlet assembly is also configured such that when the one or more second parts are removed from the first part, at least some, most or all of the first channel and/or the one or more second channels is exposed.

In particular embodiments, the nebuliser outlet assembly is configured such that when the one or more second parts are removed from the first part, at least an outlet end of the first channel and/or the one or more second channels is exposed. This may be such that when a liquid capillary is installed in the first channel (in the first part), it may be installed at a desired position within the first channel (e.g. such that a user can clearly see and/or measure this position). For example, the nebuliser outlet assembly may be configured such that when the one or more second parts are removed from the first part, a user can see and/or measure a distance between an outlet of the liquid capillary and the outlet aperture and/or the outlet end (in an axial direction that extends along a length of the nebuliser outlet assembly and/or along a length of the liquid capillary), e.g. such that the user can set this distance to a desired distance.

Thus, the nebuliser outlet assembly may be configured such that when the first part and the one or more second parts are disassembled (i.e. when the nebuliser outlet assembly is disassembled), at least some, most or all of the first channel and/or the one or more second channels are exposed (e.g. to a user of the nebuliser outlet assembly).

In embodiments, the first channel is formed from the first part and the one or more second parts when the one or more second parts are attached to the first part, and at least some, most or all of the first channel is exposed when the one or more second parts are removed from the first part.

This may be achieved in any suitable manner. For example, the first part may comprise an open-sided channel (e.g. groove) that corresponds to the first channel (as described above), and may be configured such that when the one or more second parts are removed from the first part, the open side of the channel is exposed. The channel may be open-sided for some, most or all of its length. The assembly may be configured such that the complete (tubular) first channel is formed when the one or more second parts are attached to the first part, e.g. by the one or more second parts covering the open-side of the channel.

Alternatively, the first part and the one or more second parts may each comprise complimentary open-sided channels (e.g. grooves) (that each corresponds to the first channel, as described above), where the complete (tubular) first channel is formed when the complimentary open-sided channels meet (and face one another) when the one or more second parts are attached to the first part. In these embodiments, both open sides of the complimentary open-sided channels may be exposed when the one or more second parts are removed from the first part

Configuring the first channel in this manner means that, during assembly, a user may install a liquid capillary into the first channel when the one or more second parts have been removed from the first part. This allows the user to (visually) verify and/or measure whether the liquid capillary has been properly installed into the first channel, e.g. at the correct axial position. In this way, the spatial parameters which affect the interaction between the liquid emitted by the capillary and the nebulising gas can be precisely controlled by the user. Thus, variation in the performance of the nebuliser can be significantly reduced, for example, from one use to the next (for example after uninstalling and re-installing a liquid capillary), and from one nebuliser to the next.

Furthermore, this configuration allows the first channel to be cleaned, e.g. when the one or more second parts are removed from the first part.

One or more or each of the second channel(s) may be configured in a similar manner as the first channel (although this need not be the case). Thus, one or more or each second channel may be formed from the first part and the one or more second parts when the one or more second parts are attached to the first part, and at least some, most or all of one or more or each second channel may be exposed when the one or more second parts are removed from the first part.

This may be achieved in any suitable manner. For example, the first part may comprise one or more open-sided channels (e.g. groove(s)) that may (each) correspond to the or each second channel (as described above), and may be configured such that when the one or more second parts are removed from the first part, the open side(s) of the channel(s) is exposed. The assembly may be configured such that the or each complete (tubular) second channel is formed when the one or more second parts are attached to the first part, e.g. by the one or more second parts covering the open-side of the channel.

Alternatively, the first part and the one or more second parts may each comprise complimentary open-sided channels (that may each correspond to the or each second channel, as described above), where the or each complete (tubular) second channel is formed when the complimentary open-sided channels meet (and face one another) when the one or more second parts are attached to the first part. In these embodiments, both open sides of the complimentary open-sided channels may be exposed when the one or more second parts are removed from the first part.

Configuring one or more or each second channel in this manner allows the second channel(s) to be cleaned, e.g. when the one or more second parts are removed from the first part.

As well as forming the first and/or second channel(s), the first and/or one or more second parts may also form the inlet end and/or the outlet end of the nebuliser outlet assembly.

The first and/or second part may have any suitable form. For example, the first part may extend along some, most or all of the axial length of the nebuliser outlet assembly, e.g. from the inlet end to the outlet end. Similarly, the or each second part may extend along some, most or all of the axial length of the nebuliser outlet assembly, e.g. from the inlet end to the outlet end.

The first and/or second part of the nebuliser outlet assembly may be formed using an additive manufacturing process. The use of additive manufacturing can significantly reduce manufacturing imperfections in the part, and also facilitates the creation of nebuliser outlet assembly geometries that would be impractical (or even impossible) to form using conventional subtractive machining processes.

The first and/or second part of the nebuliser outlet assembly may be formed using Selective Laser Melting (SLM). Alternatively, the first and/or second part of the nebuliser outlet assembly may be formed using electron-beam additive manufacturing.

According to an aspect, there is provided a nebuliser comprising:
the nebuliser outlet assembly described above; and
a liquid capillary;
wherein the liquid capillary is retained by the first channel (for example by the at least part of the first channel that has the second area (second diameter)).

According to an aspect, there is provided an ion source comprising the nebuliser outlet assembly and/or the nebuliser described above.

The ion source may comprise an Electrospray Ionisation (ESI) ion source, a Desorption Electrospray Ionisation (DESI) ion source, a Desorption Electro-Flow Focusing Ionisation (DEFFI) ion source, an impactor ion source, or an Atmospheric Pressure Chemical Ionisation (APCI) ion source.

According to an aspect, there is provided an analytical instrument such as a mass and/or ion mobility spectrometer, comprising the nebuliser outlet assembly and/or the nebuliser and/or the ion source described above.

According to an aspect, there is provided a method of assembling the nebuliser outlet assembly described above, the method comprising:
inserting a liquid capillary into the first channel when the one or more second parts are removed from the first part; and then
attaching the one or more second parts to the first part.

According to an aspect, there is provided a method of assembling a nebuliser outlet assembly that comprises:
a first part and one or more second parts that include an inlet end and an outlet end, and a first channel arranged between the inlet end and the outlet end, wherein the first channel is configured to receive a capillary;
the method comprising:
inserting a liquid capillary into the first channel when the one or more second parts are removed from the first part such that at least some of the first channel is exposed; and then
attaching the one or more second parts to the first part so as to form the first channel.

Inserting the liquid capillary into the first channel may comprise inserting the liquid capillary into the first channel at a desired position within the first channel.

Inserting the liquid capillary into the first channel may comprise inserting the liquid capillary into the first channel such that a distance between an outlet of the liquid capillary and the outlet aperture and/or the outlet end (in an axial direction that extends along a length of the nebuliser outlet assembly and/or along a length of the liquid capillary) is set to a desired distance.

According to an aspect, there is provided a method of nebulising a liquid, the method comprising using the nebuliser outlet assembly and/or the nebuliser described above to nebulise a liquid.

According to an aspect, there is provided a method of ionisation, the method comprising using the nebuliser outlet assembly and/or the nebuliser and/or the ion source described above to ionise an analyte.

According to an aspect, there is provided a method of analysing an analyte, the method comprising using the ion source described above to ionise an analyte so as to produce analyte ions, and analysing the analyte ion and/or ions derived from the analyte ions.

Analysing the analyte ions and/or ions derived from the analyte ions may comprise determining the mass to charge ratio, mass, charge, ion mobility and/or collision cross section of the analyte ions and/or of the ions derived from the analyte ions, for example using an analytical instrument such as a mass and/or ion mobility spectrometer.

According to an aspect, there is provided a method of manufacturing a nebuliser outlet assembly, the method comprising manufacturing the nebuliser outlet assembly described above e.g. using an additive manufacturing process.

The additive manufacturing process may comprise Selective Laser Melting (SLM). The additive manufacturing process may comprise electron-beam additive manufacturing.

### BRIEF DESCRIPTION OF THE DRAWINGS

Various embodiments will now be described, by way of example only, and with reference to the accompanying drawings in which:

FIG. 1 shows schematically a cross-sectional view of a conventional Electrospray Ionisation (ESI) ion source nebuliser;

FIG. 2(a) shows an image of a conventional Electrospray Ionisation (ESI) ion source nebuliser, FIG. 2(b) shows schematically an end view of a conventional Electrospray Ionisation (ESI) ion source nebuliser, FIG. 2(c) shows an image of a conventional Electrospray Ionisation (ESI) ion source nebuliser, and

FIG. 2(d) shows schematically an end view of a conventional Electrospray Ionisation (ESI) ion source nebuliser;

FIG. 3 shows schematically a cross-sectional view of a nebuliser assembly in accordance with various embodiments of the present invention;

FIG. 4(a) shows schematically a cross-sectional view of a first part of a nebuliser in accordance with various embodiments of the present invention, and

FIG. 4(b) shows schematically a cross-sectional view of a second part of a nebuliser in accordance with various embodiments of the present invention:

FIG. 5(a) shows an exploded view of a nebuliser assembly in accordance with embodiments of the present invention, and FIG. 5(b) shows an assembled view of a nebuliser in accordance with various embodiments of the present invention; and

FIGS. 6(a)-6(d) show a method of assembling a nebuliser in accordance with various embodiments of the present invention.

### DETAILED DESCRIPTION

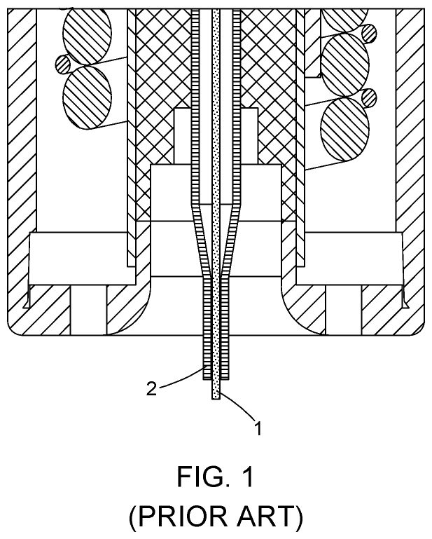

FIG. 1 shows schematically a cross sectional view of a conventional nebuliser of an Electrospray Ionisation (ESI) ion source. As shown in FIG. 1, the nebuliser comprises a liquid capillary 1 and a gas capillary 2. The liquid capillary 1 is arranged coaxially within the gas capillary 2, with the liquid-emitting outlet of the liquid capillary 1 extending beyond the distal end of the gas capillary 2. A flow of liquid supplied to the liquid capillary 1 is nebulised by a nebulising gas flow supplied to the gas capillary 2.

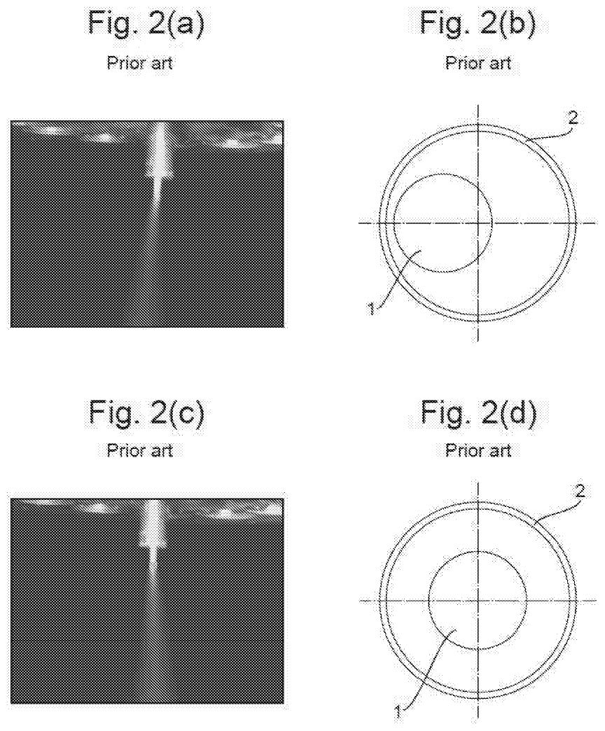

FIGS. 2(b) and 2(d) show an end view of the conventional Electrospray Ionisation (ESI) ion source nebuliser. As can be seen in FIGS. 2(b) and 2(d), a gap between the outer diameter of the liquid capillary 1 and the inner diameter of the gas capillary 2 creates an annulus through which the nebulising gas passes.

In this conventional design, the position of the liquid capillary 1 within the gas capillary 20 is not directly restrained by the gas capillary 2. Instead, the gas flow is relied upon to force the liquid capillary 1 into a central position within the gas capillary 2.

As shown in FIGS. 2(c) and 2(d), when the liquid capillary 1 sits centrally within the gas capillary 2, an equal gas flow within the annulus creates an evenly distributed spray.

However, the Applicant has recognised that it is possible for the liquid capillary 1 to sit to one side of the gas capillary 2. This may be due, for example, to manufacturing imperfections in the liquid capillary 1 (or the gas capillary 2) which mean that it may not be completely straight.

As shown in FIGS. 2(a) and 2(b), the Applicant has also recognised that when the liquid capillary 1 sits to one side of the gas capillary 2, an uneven gas flow within the annulus creates an uneven spray. This can lead to undesired variation in performance of the nebuliser, and therefore in the ion source and the analytical instrument.

Various embodiments are directed to a nebuliser outlet assembly (a nebuliser outlet assembly tip) of a nebuliser, such as a nebuliser of an ion source such as an Electrospray Ionisation (ESI) ion source.

The nebuliser outlet assembly has an inlet end, and an outlet end that has an outlet aperture. A first channel and one or more second channels are arranged between the inlet end and the outlet end.

The first channel may be coaxially aligned (or non-coaxially aligned) with the outlet aperture. At least part of the first channel may optionally have an area (a cross-sectional area) that is less than the (cross-sectional) area of the outlet aperture. For example, at least part of the first channel may have a diameter that is less than the diameter of the outlet aperture. The first channel may be configured in this manner so as to retain (the outlet of) a liquid capillary relative to (such as centrally to and/or in coaxial alignment with) the outlet aperture when the liquid capillary is installed in the nebuliser outlet assembly.

The one or more second channels may be configured to pass gas from a gas inlet (which may optionally be arranged at the inlet end) to the outlet end, such as to the outlet aperture. The nebuliser outlet assembly may be configured in this manner such that, when the liquid capillary is installed in the nebuliser outlet assembly, gas received at the inlet end is passed to the outlet end via the one or more second channels, and is emitted from the outlet end such as from the outlet aperture (optionally through an annulus between the liquid capillary and the outlet aperture) so as to nebulise liquid emitted from the outlet of the liquid capillary.

As will be described in more detail below, various embodiments provide a nebuliser outlet in which a liquid capillary can be retained relative to (centrally to) the outlet aperture while at the same time allowing a nebulising gas to be provided to the outlet aperture.

Retaining the liquid capillary relative to (centrally to) the outlet aperture in this manner means that the nebuliser can produce an evenly distributed spray, and can significantly reduce variation in the performance of the nebuliser, for example from one use to the next (for example after uninstalling and re-installing a liquid capillary), and from one nebuliser to the next.

Furthermore the nebuliser outlet assembly is formed from (at least) a first part and one or more second parts, where the one or more second parts are removably attachable to the first part. The nebuliser outlet assembly is configured such that when the one or more second parts are attached to the first part (i.e. when the nebuliser outlet assembly is assembled), the first channel (and optionally the one or more second channels) is formed from the first part and from the one or more second parts. This may such that when assembled (i.e. when the one or more second parts are attached to the first part), most or all of the first channel (and optionally most or all of one or more second channels) (e.g. except its inlet(s) and/or outlet(s)) is substantially concealed (e.g. from a user of the nebuliser outlet assembly).

The nebuliser outlet assembly is also configured such that when the one or more second parts are removed from the first part (i.e. when the nebuliser outlet assembly is disassembled), at least some of the first channel (and optionally the one or more second channels) is exposed (e.g. to a user of the nebuliser outlet assembly).

This configuration means that, during assembly, a user may install a liquid capillary into the (first channel of the) nebuliser outlet assembly when the one or more second parts have been removed from the first part. This allows the user e.g. to (visually) verify and/or measure whether the liquid capillary has been properly installed into the (first channel of the) nebuliser outlet assembly.

This then means that spatial parameters of the nebuliser outlet assembly, in particular those parameters which affect the interaction between the liquid emitted by the capillary and the nebulising gas, can be precisely controlled and/or substantially fixed. In particular, a user can see and precisely set the distance between an outlet of the liquid capillary and the outlet aperture and/or the outlet end (in an axial direction that extends along a length of the nebuliser outlet assembly and/or along a length of the liquid capillary), which is an important parameter which affects the interaction between the liquid emitted by the capillary and the nebulising gas. Thus, variation in the performance of the nebuliser can be significantly reduced, for example, from one use to the next (for example after uninstalling and re-installing a liquid capillary), and from one nebuliser to the next.

Furthermore, embodiments facilitate cleaning of the internal features (i.e. the first and/or one or more second channels) of the nebuliser outlet assembly, e.g. by providing access to the internal features (i.e. the first and/or one or more second channels) of the nebuliser outlet assembly, e.g. when the one or more second parts are removed from the first part.

Various embodiments accordingly provide an improved nebuliser outlet assembly.

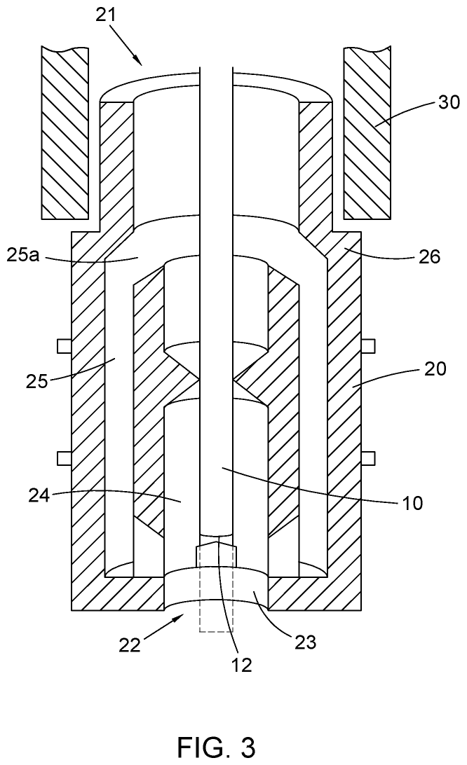

FIG. 3 shows schematically a cross-sectional view of a nebuliser assembly in accordance with various embodiments.

As shown in FIG. 3, the nebuliser assembly comprises a nebuliser outlet assembly 20 which has an inlet end 21 and an outlet end 22. The nebuliser assembly 20 may have a central axis, which may extend between the inlet end 21 and the outlet end 22 in an axial direction. A radial direction may extend outwardly from the central axis.

The nebuliser outlet assembly 20 may be configured to emit a spray of droplets, generally in the axial direction, such as a spray of nebulised droplets, from its outlet end 22. To do this, the nebuliser outlet assembly 20 may be configured to receive at its inlet end 21 a flow of liquid and a flow of gas, and to cause the liquid to be nebulised by the gas so as to produce the spray of droplets.

The nebuliser outlet assembly 20 may be configured to receive the flow of gas from a tube 30 such as a gas supply tube which may be connected to the nebuliser outlet assembly 20. The inlet end 21 of the nebuliser outlet assembly 20 may be configured to be attached to the tube 30, for example in a gas tight manner, such that gas provided to an inlet of the tube 30 is provided to the inlet end 21 of the nebuliser outlet assembly 20.

An outlet end of the tube 30 may be configured to surround (or to be surrounded by) the inlet end 21 of the nebuliser outlet assembly 20 when the nebuliser outlet assembly 20 is attached to the tube 30, for example such that the nebuliser outlet assembly 20 can be attached to the tube 30 in a gas tight manner. The nebuliser outlet assembly 20 can be attached to the tube 30 in any suitable manner, such as for example, using an interference fit, weld, glue, and the like, between the outlet end of the tube 30 and the inlet end 21 of the nebuliser outlet assembly 20.

The nebuliser outlet assembly 20 may be configured to receive the flow of liquid via a liquid capillary 10. The liquid capillary 10 may comprise an inlet end and an outlet end 12, and may be configured such that liquid provided to the inlet is emitted from the outlet end 12.

The liquid capillary 10 may have any suitable configuration, such as for example, the configuration described in WO 2015/040384, the content of which is incorporated herein by reference.

The liquid capillary 10 may have any suitable outer diameter, such as for example (i)<0.1 mm; (ii) 0.1-0.15 mm; (iii) 0.15-0.2 mm; (iv) 0.2-0.25 mm; (v) 0.25-0.3 mm; or (vi) >0.3 mm. In various particular embodiments, the liquid capillary 10 has an outer diameter of approximately 0.22 mm.

The liquid capillary 10 may be formed from an electrically conductive material such as a metal such as stainless steel. In embodiments, a voltage such as a high voltage may be applied to the liquid capillary 10, for example such that the spray of droplets emitted by the nebuliser outlet assembly 20 comprises a spray of charged droplets, for example in the manner of an Electrospray Ionisation (ESI) ion source.

The nebuliser outlet assembly 20 may be configured to receive and retain (in place) the liquid capillary 10. When installed in the nebuliser outlet assembly, the liquid capillary 10 may pass through the centre of the tube 30 and through the centre of the nebuliser outlet assembly 20. That is, the liquid capillary 10 may be arranged along the central axis of the nebuliser outlet assembly 20. However, it would also be possible for the liquid capillary 10 to be arranged in a non-concentric manner, such as parallel to and radially spaced from the central axis of the nebuliser outlet assembly 20.

The outlet end 22 of the nebuliser outlet assembly 20 comprises an outlet aperture 23. The outlet aperture 23 may be arranged on the central axis of the nebuliser outlet assembly 20. However, it would also be possible for the outlet aperture 23 to be arranged in a non-coaxial manner, such as off-axis from the central axis of the nebuliser outlet assembly 20.

As shown in FIG. 3, the nebuliser outlet assembly 20 may be configured such that, when the liquid capillary 10 is installed in the nebuliser outlet assembly 20, the outlet (tip) 12 of the liquid capillary 10 passes through and protrudes beyond the outlet aperture 23. The outlet 12 of the liquid capillary 10 may protrude beyond the outlet aperture 23 by any suitable distance, such as for example by around (i) 0-0.5 mm; (ii) 0.5-1.0 mm; (iii) 1.0-1.5 mm; or (iv) >1.5 mm.

Alternatively, the nebuliser outlet assembly 20 may be configured such that, when the liquid capillary 10 is installed in the nebuliser outlet assembly 20, the outlet (tip) 12 of the liquid capillary 10 does not protrude beyond the outlet aperture 23, but is instead arranged within the outlet assembly 20, upstream of the outlet aperture 23. In these embodiments, the outlet 12 of the liquid capillary 10 may be arranged upstream of the outlet aperture 23 by any suitable distance, such as for example a distance up to 0.5 mm.

As described elsewhere herein, this distance is an important parameter that affects the nature of the spray emitted by the nebuliser. Embodiments allow this distance to be precisely controlled and set by a user when installing the liquid capillary 10 into the nebuliser outlet assembly 20.

The nebuliser outlet assembly 20 may optionally be configured such that when the liquid capillary 10 is installed in the nebuliser outlet assembly 20, an annulus is formed between the outer diameter of the liquid capillary 10 and the inner diameter of the outlet aperture 23. The nebuliser outlet assembly 20 may be configured such that, when the liquid capillary 10 is installed in the nebuliser outlet assembly 20, gas received at the inlet end 21 is emitted from the outlet aperture 23 through the annulus, so as to nebulise liquid emitted from the outlet 12 of the liquid capillary 10.

The outlet aperture 23 may have a first cross-sectional area, which may be larger than a cross-sectional area of the liquid capillary 10. For example, the outlet aperture 23 may have a first inner diameter which may be larger than an outer diameter of the liquid capillary 10. The outlet aperture 23 may have any suitable inner diameter, which may be larger than the outer diameter of the liquid capillary 10, such as for example (i)<0.2 mm; (ii) 0.2-0.25 mm; (iii) 0.25-0.3 mm; (iv) 0.3-0.35 mm; (v) 0.35-0.4 mm; or (vi) >0.4 mm. In embodiments, the outlet aperture 23 has an inner diameter of approximately 0.31 mm.

The nebuliser outlet assembly 20 comprises a first internal channel 24 arranged between the inlet end 21 and the outlet end 22. The first channel 24 may be arranged to extend along the central axis of the nebuliser outlet assembly 20 (or may be off-axis).

The first channel 24 may be configured to receive the liquid capillary 10 when the liquid capillary 10 is installed in the nebuliser outlet assembly 20. The first channel 24 may be (e.g. coaxially) aligned with the outlet aperture 23, for example such that when the liquid capillary 10 is installed in the nebuliser outlet assembly 20, the liquid capillary 10 passes through the first channel 24 (and optionally also passes through and protrudes beyond the outlet aperture 23).

The first channel 24 may also be configured to retain the liquid capillary 10, i.e. to hold the liquid capillary in place, relative to the nebuliser outlet assembly 20, at least in the radial direction. In particular, the first channel 24 may be configured such that when the liquid capillary 10 is installed in the nebuliser outlet assembly 20, the first channel 24 retains the outlet of the liquid capillary 10 centrally to, such as in coaxial alignment with, the outlet aperture 23.

To do this, at least part of the first channel 24 may have a second cross-sectional area that is less than the first cross-sectional area of the outlet aperture 23. For example, at least part of the first channel 24 may have a second inner diameter that is less than the first inner diameter of the outlet aperture 23. The second area may be approximately equal to or slightly larger than the cross-sectional area of the liquid capillary 10, and the second diameter may be approximately equal to or slightly larger than the outer diameter of the liquid capillary 10, such that the outlet of the liquid capillary 10 is retained centrally to and/or in coaxial alignment with the outlet aperture 23 when the liquid capillary 10 is passed through the first channel 24.

The second diameter may be, for example, around (i)<0.1 mm; (ii) 0.1-0.15 mm; (iii) 0.15-0.2 mm; (iv) 0.2-0.25 mm; (v) 0.25-0.3 mm; or (vi) >0.3 mm. In embodiments, the at least part of the first channel 24 has a second inner diameter of approximately 0.25 mm.

In embodiments, the first channel 24 may have the same cross-sectional area (i.e. the same inner diameter) along its entire axial length. In these embodiments, the first channel 24 may be a tube such as a generally cylindrical tube having the second cross sectional area (i.e. second diameter), which may be arranged to extend along the central axis of the nebuliser outlet assembly 20.

However, in embodiments, as shown in FIG. 3, the first channel 24 may have plural different cross sectional areas (i.e. plural different inner diameters) along its axial length, where at least part of the first channel 24 has the second cross sectional area (i.e. second diameter) that is less than the first cross sectional area (i.e. first diameter of the outlet aperture 23).

For example, as shown in FIG. 3, the first channel 24 may comprise a first part that has the second cross sectional area (i.e. second diameter) and one or more second, e.g. tubular, parts that may have a cross sectional area (i.e. diameter) that is greater than the second cross sectional area (i.e. second diameter). Other arrangements are possible, e.g., as described elsewhere herein. The part of the first channel 24 that has the second cross sectional area (second diameter) may have any suitable length and may be located at any suitable position relative to the outlet end 22 of the nebuliser outlet assembly 20.

The nebuliser outlet assembly 20 comprises one or more second (internal) channels 25 arranged between the inlet end 21 and the outlet end 22. Each of the one or more second channels 25 may be arranged to run alongside the central axis of the nebuliser outlet assembly 20, such as being (at least in part) parallel to and spaced apart from the central axis of the nebuliser outlet assembly 20 (and so parallel to the first channel 24). However, one or more or each second channel need not be entirely or precisely parallel to the central axis. In general, one or more or each second channel may have any suitable configuration such as a tapered configuration, zig-zag configuration, helix configuration, matrix configuration, and the like.

The one or more second channels 25 are configured to pass gas from the inlet end 21 to the outlet aperture 23. In other words, the one or more second channels 25 are configured such that gas received at the inlet end 21 is passed to the outlet aperture 23 via the one or more second channels 25 (and bypasses the first channel 24).

In particular, the one or more second channels 25 may be configured such that when the liquid capillary 10 is installed in the nebuliser outlet assembly 20, gas received at the inlet end 21 is passed to the outlet end 22 via the one or more second channels 25, and is emitted from the outlet end such as from the aperture 23 (optionally through an annulus between the liquid capillary 10 and the outlet aperture 23) so as to nebulise liquid emitted from the outlet of the liquid capillary 10.

To do this, an inlet end of each second channel 25 may be arranged at or close to the inlet end 21 of the nebuliser outlet assembly 20, and may be configured to receive gas, for example, from the gas supply tube 30. An outlet end of each second channel 25 may be arranged at or close to the outlet end 22 of the nebuliser outlet assembly 20, and may be in fluid communication with the outlet aperture 23, such that gas received at the inlet end of the channel is provided to and emitted from the outlet aperture 23.

Since, as described above, the first part of the first channel 24 may have a cross-sectional area (e.g. diameter) that is similar to (e.g. equal to or slightly larger than) the cross-sectional area (e.g. outer diameter) of the capillary 10, most or all of the gas received by the outlet 20 will be forced to pass to the outlet end 22 via the one or more second channel(s) 25.

Alternatively, an inlet end of one or more or each second channels 25 may be arranged at some other point along the length of the nebuliser outlet assembly 20.

Each second channel may have any suitable form. For example, each second channel may be a tube such as a generally cylindrical tube, which may be arranged to extend parallel to the central axis of the nebuliser outlet assembly 20. Other cross section shapes would be possible.

The nebuliser outlet assembly 20 may comprise any suitable number of second channels. For example, the nebuliser outlet assembly 20 may comprise a single second channel, or plural second channels, such as two, three, four, five or more second channels.

In various embodiments, the one or more second channels 25 comprises an annular channel. In these embodiments, an outlet end of the annular channel may be in fluid communication with the outlet aperture 23, for example via one or more channels which may be extend in the radial direction. This arrangement has been found to improve reproducibility with respect to the manufacturing process. In particular, an annular channel is much less susceptible to being blocked by excess powder during the additive manufacturing (e.g. SLM) process, when compared with individual tubular channels (which can become blocked with excess powder).

The width of the annular channel may be selected so that the wall between the annular channel and the first channel 24 provides mechanical stability, while also allowing sufficient gas flow through nebuliser outlet assembly 20 via the second channel 25.

In various embodiments, the one or more second channels 25 may comprise a segmented annular channel. Thus, the cross-sectional shape each of the one or more second channels 25 may (approximately) correspond to an annular sector (annulus sector). The segmented annular channel 25 may coaxially surround the central first channel 24 for some or most of the length of the nebuliser outlet assembly 20, but may converge with the first channel 24 at a convergence region close to the outlet aperture 23.

The cross-sectional shape of one or more or each of the one or more second channels 25 may be constant or may change along the length of the one or more second channels 25. The annular channel can be segmented into any suitable number of channels, such as two, three, four, five, etc. channels.

This arrangement has been found to provide robust mechanical stability, while improving reproducibility with respect to the manufacturing process. In particular, the material between each segment will provide additional mechanical support, while a segmented annular channel is much less susceptible to being blocked by excess powder during the additive manufacturing (e.g. SLM) process, when compared with individual tubular channels.

The width of the segmented annular channels may be selected so that the wall between the segmented annular channels and the first channel 24 provides mechanical stability, while also allowing sufficient gas flow through nebuliser outlet assembly 20 via the segmented annular channels.

Where there are plural second channels, each channel may be substantially identical (although this need not be the case), and may for example, be arranged in a rotationally symmetric configuration about the central axis of the nebuliser outlet assembly 20, although this need not be the case.

Configuring the nebuliser outlet assembly 20 to have one or more second channels 25 in addition to the first channel 24 in this manner means that the liquid capillary 10 can be retained relative to (such as centrally to) the outlet aperture 23 while at the same time allowing a nebulising gas to be provided to the outlet end 22 such as to the outlet aperture 23 via the one or more second channels 24. This means that the nebuliser outlet assembly 20 can produce an evenly distributed spray, and can significantly reduce variation in the performance of the nebuliser outlet assembly 20, for example from one use to the next (for example after uninstalling and re-installing a liquid capillary 10), and from one nebuliser outlet assembly 20 to the next.

As also shown in FIG. 3 the nebuliser outlet assembly 20 may comprise one or more overhangs 26 (protrusions) arranged adjacent to an inlet or inlets 25a of the one or more second channels 25. The one or more overhangs 26 (protrusions) may extend radially inwardly, and may be positioned and configured to prevent snagging of the liquid capillary 10, for example so as to prevent snagging of the liquid capillary 10 in the inlet(s) 25a of the one or more second channels 25, when the liquid capillary 10 is installed in the nebuliser outlet assembly 20. This may facilitate more straightforward installation of the liquid capillary 10 in the nebuliser outlet assembly 20. For example, as shown in FIG. 3, in various embodiments, the one or more overhangs or protrusions 26 comprise a reduced cross sectional area (reduced inner diameter) portion in the first channel 24.

In various embodiments, the nebuliser outlet assembly 20 may be configured such that, when the liquid capillary 10 is installed in the nebuliser outlet assembly 20, the outlet (tip) 12 of the liquid capillary 10 does not protrude beyond the outlet aperture 23, but is instead arranged within the outlet 20, upstream of the outlet aperture 23. This arrangement may produce so-called “flow blurring” nebulisation, i.e. where highly turbulent mixing between the liquid emitted from the capillary 10 and the nebulising gas flow creates a fine aerosol of extremely small droplets.

In these embodiments (and other embodiments described herein), the nebuliser may be configured such that the distance between the outlet 12 of the capillary 10 and the outlet aperture 23 is adjustable (controllable), e.g. so that the properties of the spray that depend on this distance can be controlled (in use). For example, the nebuliser may include a mechanical arrangement, such as the mechanical arrangement described in GB 2562168 (Micromass UK Ltd), the contents of which are incorporated herein by reference, which may be configured to allow the distance between the outlet 12 of the capillary 10 and the outlet aperture 23 to be adjusted. Other arrangements would, however, be possible.

As described above, the nebuliser outlet assembly 20 is formed from (at least) a first part and one or more second parts, where the one or more second parts are removably attachable to the first part. The nebuliser outlet assembly 20 is configured such that when the one or more second parts are attached to the first part (i.e. when the nebuliser outlet assembly is assembled), the first channel 24 (and optionally the one or more second channels 25) is formed from the first part and from the one or more second parts. This may such that when assembled, i.e. when the one or more second parts are attached to the first part, most or all of the first channel 24 and optionally most or all of one or more second channels 25 (e.g. except its inlet(s) and/or outlet(s)) is substantially concealed (e.g. from a user of the nebuliser outlet assembly 20).

The nebuliser outlet assembly 20 is also configured such that when the one or more second parts are removed from the first part (i.e. when the nebuliser outlet assembly is disassembled), at least some of the first channel 24 and optionally the one or more second channels 25 is exposed (e.g. to a user of the nebuliser outlet assembly).

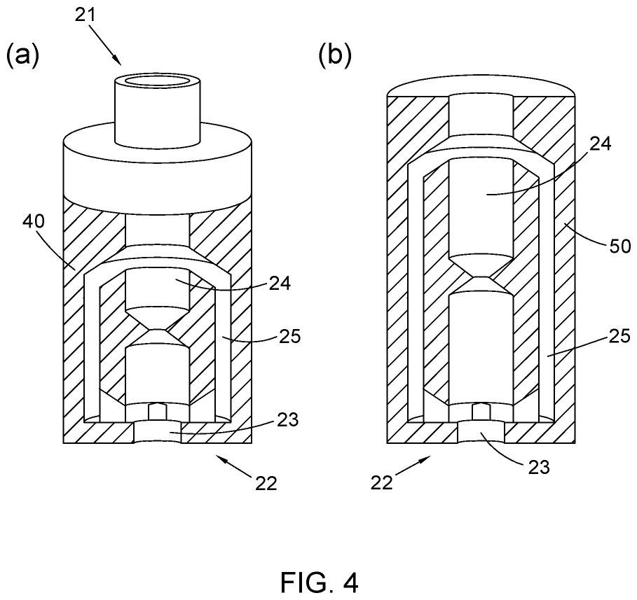

FIG. 4(a) shows schematically a cross-sectional view of a first part 40 of a nebuliser in accordance with various embodiments, and FIG. 4(b) shows schematically a cross-sectional view of a corresponding second part 50 of a nebuliser in accordance with various embodiments. The second part 50 is removably attachable to the first part 40.

As shown in FIGS. 4(a) and 4(b), the nebuliser outlet assembly 20 is configured such that the first channel 24 and/or the one or more second channels 25 are formed from the first part 40 and from the second part 50 when the second part 50 is attached to the first part 40. That is, when the first part 40 and the second part 50 are assembled together, the first channel 24 and/or the one or more second channels 25 are formed from the first part 40 and from the second part 50. This is such that when assembled, most or all of the first channel 24 and/or the one or more second channels 25 (e.g. except their inlet(s) and/or outlet(s)) are substantially concealed (e.g. from a user of the nebuliser outlet assembly).

As also shown in FIGS. 4(a) and 4(b), the nebuliser outlet assembly 20 is configured such that when the second part 50 is removed from the first part 40, at least some, most or all of the first channel 24 and/or the one or more second channels 25 is exposed. This is such that when a liquid capillary 10 is installed in the first channel 24 (in the first part 40), it can be installed at a desired position within the first channel 24, such that a user can clearly see and/or measure this position. In particular, a user can see and/or measure a distance between the outlet 12 of the liquid capillary 10 and the outlet aperture 23 and/or the outlet end 22 (in the axial direction), such that the user can set this distance to a desired value.

As shown in FIGS. 4(a) and 4(b) the first part 40 and the second part 50 each comprise complimentary open-sided channels (e.g. grooves) (that each corresponds to the first channel 24 and/or second channels 25), where the complete (tubular) first channel 24 and/or second channel(s) 25 are formed when the complimentary open-sided channels meet (and face one another) when second part 50 is attached to the first part 40. Both open sides of the complimentary open-sided channels are exposed when the second part 50 is removed from the first part 40.

During assembly, a user may install a liquid capillary 10 into the first channel 24 when the second part 50 has been removed from the first part 50. This allows the user to (visually) verify and/or measure whether the liquid capillary 10 has been properly installed into the first channel 24 at the correct axial position. In this way, the distance, which affects the interaction between the liquid emitted by the capillary and the nebulising gas can be precisely controlled by the user. Thus, variation in the performance of the nebuliser can be significantly reduced, for example, from one use to the next (for example after uninstalling and re-installing a liquid capillary), and from one nebuliser to the next.

Furthermore, this configuration allows the first channel to be cleaned, e.g. when the second part 50 is removed from the first part 40.

As show in FIGS. 4(a) and 4(b), the first part 40 may be arranged between the inlet end 21 and the outlet end 22. In particular, the first part 40 may extend along the entire length of the nebuliser outlet assembly 20 between the inlet end 21 and the outlet end 22 (although it would be possible for the first part 40 to extend along only part (some but not all) of the length of the nebuliser outlet assembly 20 between the inlet end 21 and the outlet end 22).

The first part 40 may in effect have a cut away section, that may extend along some, most or all of the length of the nebuliser outlet assembly 20, e.g. at the outlet end 22. The second part 50 may have a size, shape, and/length etc. that compliments the cut away section of the first part 40. Thus, the second part 50 may extend along some, most or all of the entire of the nebuliser outlet assembly 20 between the inlet end 21 and the outlet end 22. When the second part 50 is attached to the first part 40, it may in effect fill the cut away section of the first part 40.

The first part 40 and the second part 50 are configured to form the first channel 24 when the second part 50 is removably attached to the first part 40. Similarly, the first part 40 and the second part 50 may be configured to form the one or more second channels 25, e.g. when the second part 50 is removably attached to the first part 40.

When the second part 50 is attached to the first part 40, the first channel 24 and/or the one or more second channels 25 are concealed, e.g. along the axial length of the nebuliser outlet assembly 20, by the first part 40 and the second part 50. When the second part 50 is removed from the first part 40, the first channel 24 and/or the one or more second channels 25 are (partially) exposed, e.g. along the axial length of the nebuliser outlet assembly 20.

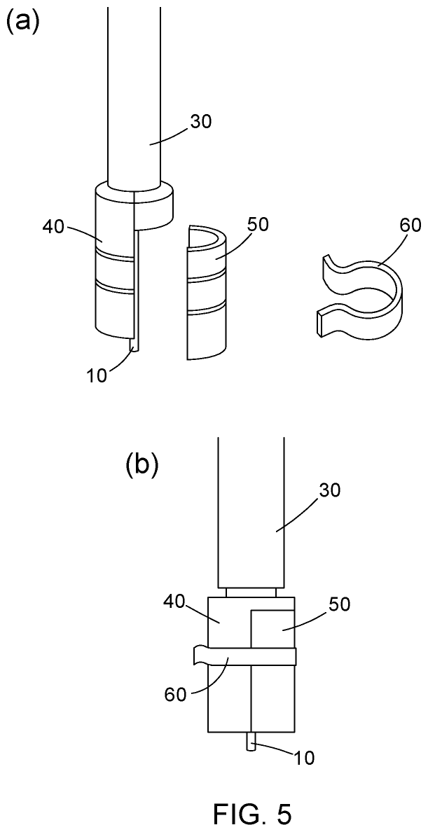

FIG. 5(a) shows an exploded view of a disassembled nebuliser assembly in accordance with various embodiments, and FIG. 5(b) shows an assembled nebuliser in accordance with various embodiments.

As shown in FIG. 5(a), the nebuliser outlet assembly 20 may comprise a clip configured to removably attach the second parts 50 to the first part 40. As shown in FIG. 5(b), the second part 50 may be removably attached to the first part 40 by suitably positioning the clip 60 around the first part 40 and the second part 50.

More generally, the one or more second parts 50 may be removably attachable to the first part 40 using any suitable means. For example, the first part 40 and/or the one or more second parts 50 may (each) include a suitable attachment fitting(s) to allow the one or more second parts to be removably attached to the first part. Alternatively, the nebuliser outlet assembly 20 may include one or more (separate) attachment fittings, such as for example one or more clamps, one or more clips, one or more screw fittings, one or more straps, and the like, configured to allow the one or more second parts 50 to be removably attached to the first part 40.

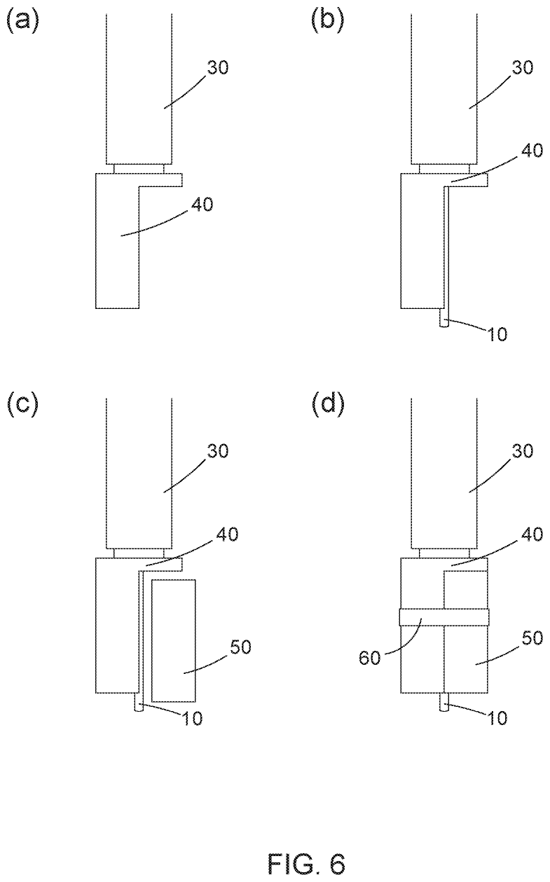

FIGS. 6(a)-6(d) illustrate a method of assembling a nebuliser in accordance with various embodiments.

As shown in FIG. 6(a), the first part 40 may be fixedly attached to the gas tube 30. However, it would also be possible for the first part 40 to be separate from the tube 30, i.e. the first part 40 may be removable. In this latter case, a user may (removably) attach the first part 40 to the tube 30.

The user may then insert (and position) a liquid capillary 10 into the nebuliser outlet assembly 20, as shown in FIG. 6(b). In particular, the user may insert (and position) the liquid capillary 10 in a first channel 24 (not shown) of the first part 40 of the nebuliser outlet assembly 20. To do this, the liquid capillary 10 may be passed through the tube 30.

As shown in FIG. 6(c), once the user has verified that the liquid capillary 10 has been correctly inserted (and positioned) within the nebuliser outlet assembly 20, the user may bring the first part 40 and the second part 50 together.

The user may then (removably) attach the second part 50 to the first part 40, i.e. by positioning a clamp 60 around the first part 40 and the second part 50, as shown in FIG. 6(d).

Although various embodiments have been described above where incoming gas is received at the inlet end 21 of the nebuliser outlet assembly 20, in various embodiments this need not be the case. In general, the nebuliser outlet assembly 20 may have one or more gas inlets at the inlet end 21 or at any point along its length. For example, the nebuliser outlet assembly 20 may have one or more gas inlets at a point between the inlet end 21 and the outlet end 22. Other arrangements would be possible.

It will be appreciated that various embodiments provide a multi-part (optionally coaxial) nebuliser outlet assembly 20 which facilitates reduced variation in performance from one use to the next (for example after uninstalling and re-installing a liquid capillary 10 and/or between repeat experimental runs), and from one nebuliser to the next.

The nebuliser outlet assembly 20 may be configured to position a fluid capillary 10 coaxially (or non-coaxially) to a gas flow to generate a consistent fine droplet spray with repeatable droplet size and distribution, where the capillary 10 may be held stably by a dedicated capillary hole 24 separate to the gas flow. Holding the capillary central to the gas flow is achieved by physical restraint of the capillary, and additional open passages around those restraints are provided to allow the gas to flow and optionally to create an annulus at the capillary tip.

The nebuliser outlet assembly 20 is formed from multiple parts such that, during assembly, a user can install the liquid capillary 10 into the (first channel of the) nebuliser outlet assembly when the second part 50 has been removed from the first part 40. This allows the user to (visually) verify and/or measure whether the liquid capillary 10 has been properly installed into the (first channel of the) nebuliser outlet assembly 20.

This then means that spatial parameters of the nebuliser outlet assembly, in particular those parameters which affect the interaction between the liquid emitted by the capillary and the nebulising gas, can be precisely controlled and/or substantially fixed. Thus, variation in the performance of the nebuliser can be significantly reduced, for example, from one use to the next (for example after uninstalling and re-installing a liquid capillary), and from one nebuliser to the next.

Furthermore, embodiments facilitate cleaning of the internal features (i.e. the first and/or one or more second channels) of the nebuliser outlet assembly, e.g. by providing access to the internal features (i.e. the first and/or one or more second channels) of the nebuliser outlet assembly, e.g. when the one or more second parts are removed from the first part.

The nebuliser outlet assembly 20 may be formed using an additive manufacturing process. The nebuliser outlet assembly 20 may be formed from any suitable material such as a metal. Thus, the nebuliser outlet assembly 20 may be formed using a metal additive manufacturing process.

In particular, the Applicant has found that the nebuliser outlet assembly 20 can be formed with appropriate dimensions (as described above) and precision using selective laser melting (SLM). Thus, in various particular embodiments, the nebuliser outlet assembly 20 is formed using selective laser melting (SLM).

Selective laser melting (SLM) is an additive manufacturing process that uses a laser to melt and fuse metallic particles, which when solidified form layers of the part. A layer of powder is applied to a build platform, and the powder is selectively fused by a laser beam. The build platform is then lowered, and the procedure is repeated so as to build up layers of the part one by one. Selective laser melting (SLM) allows highly complex parts to be manufactured, and without the need for specific tooling.

The skilled person will understand that “selective laser melting” (“SLM”) is known by a number of other names, such as for example (i) direct metal laser melting (DMLM); (ii) laser powder fusion (LPF); (iii) laser powder bed fusion (LPBF); (iv) laser sintering; (v) micro laser sintering; (vi) selective laser sintering (SLS); (vii) laser deposition; (viii) laser cladding; (ix) direct metal deposition (DMD); (x) laser metal deposition (LMD); (xi) direct metal laser deposition (DMLD); (xii) direct metal casting (DMC); and so on. As used herein the term “selective laser melting” is intended to encompass all such processes.

Various other metal additive manufacturing processes could instead be used to form the nebuliser outlet assembly 20. In particular, the nebuliser outlet assembly 20 may be formed using electron-beam additive manufacturing (also known as electron-beam melting (EBM)). Electron-beam additive manufacturing is an additive manufacturing process that works in a similar manner to selective laser melting (SLM), but that uses an electron beam to melt and fuse metallic particles.

In particular embodiments, the nebuliser outlet assembly 20 is formed entirely by an additive manufacturing process. In alternative embodiments the nebuliser outlet assembly 20 may be formed using a combination of an additive manufacturing process with a (more conventional) subtractive manufacturing process. It would also be possible to form the nebuliser outlet assembly 20 using (only) a (conventional) subtractive manufacturing process.

The nebuliser may form part of an ion source, such as an Electrospray Ionisation (ESI) ion source. It would also be possible for the nebuliser to form part of another type of ion source that utilises a nebuliser, such as for example a Desorption Electrospray Ionisation (DESI) ion source, a Desorption Electro-Flow Focusing Ionisation (DEFFI), an impactor ion source, or an Atmospheric Pressure Chemical Ionisation (APCI) ion source.

The ion source may form part of or may be connectable to an analytical instrument, such as a mass and/or ion mobility spectrometer. Ions generated by the ion source may be analysed by the analytical instrument, e.g. so as to determine one or more physico-chemical properties of the ions, such as their mass, mass to charge ratio, ion mobility, etc.

The nebuliser is configured to emit a spray of droplets, such as a spray of nebulised droplets. To do this, the nebuliser is configured to receive a flow of liquid and a flow of gas, and to cause the liquid to be nebulised by the gas so as to produce the spray of droplets.

The flow of liquid may be a flow of solvent optionally containing analyte. The liquid may be provided to the nebuliser with a flow rate of, for example, (i) 100 μL/min; (ii) 200 μL/min; (iii) 300 μL/min; (iv) 400 μL/min; or (v) 500 μL/min. The flow of liquid may be, for example, an eluent from a liquid chromatography system. Thus, the nebuliser may be coupled to a liquid chromatography or other separation device. Alternatively, the flow of liquid may be from a (sample) reservoir.

The gas may be any suitable nebulising gas such as for example nitrogen. The gas may be provided to the nebuliser with a flow rate of, for example, (i)<100 L/hr; (ii) 100-150 L/hr; (iii) 150-200 L/hr; (iv) 200-250 L/hr; (v) 250-300 L/hr; (vi) 300-350 L/hr; (vii) 350-400 L/hr; or (viii) >400 L/hr.

In some embodiments, a voltage such as a high voltage may be applied to the nebuliser and/or liquid capillary, for example such that the spray of droplets emitted by the nebuliser comprises a spray of charged droplets, for example in the manner of an Electrospray Ionisation (ESI) ion source.

Although the present invention has been described with reference to preferred embodiments, it will be understood by those skilled in the art that various changes in form and detail may be made without departing from the scope of the invention as set forth in the accompanying claims.

1. A nebuliser outlet assembly comprising:
an inlet end and an outlet end; and
a first channel arranged between the inlet end and the outlet end, wherein the first channel is configured to receive a capillary;
wherein the nebuliser outlet assembly comprises a first part and one or more second parts, wherein the one or more second parts are removably attachable to the first part;
wherein the nebuliser outlet assembly is configured such that the first channel is formed from the first part and the one or more second parts when the one or more second parts are attached to the first part; and
wherein the nebuliser outlet assembly is configured such that when the one or more second parts are removed from the first part, at least some of the first channel is exposed.

2. The nebuliser outlet assembly of claim 1, further comprising one or more second channels arranged between the inlet end and the outlet end, wherein the nebuliser outlet assembly is configured such that gas received by the nebuliser outlet can pass to the outlet end via the one or more second channels.

3. The nebuliser outlet of claim 1, wherein the nebuliser outlet is configured such that when a capillary is received by the first channel, gas provided to the nebuliser outlet can pass via the one or more second channels to the outlet end, and can nebulise liquid emitted by the capillary.

4. The nebuliser outlet assembly of claim 2, wherein:
the nebuliser outlet assembly is configured such that the one or more second channels are formed from the first part and the one or more second parts when the one or more second parts are attached to the first part; and/or
the nebuliser outlet assembly is configured such that when the one or more second parts are removed from the first part, at least some of the one or more second channels is exposed.

5. The nebuliser outlet assembly of claim 1, further comprising one or more components configured to secure the one or more second parts to the first part.

6. The nebuliser outlet of claim 1, wherein the nebuliser outlet comprises an outlet aperture arranged at the outlet end, and wherein the first channel is aligned with the outlet aperture; and
wherein the nebuliser outlet is configured such that when a capillary is received by the first channel, the capillary is retained relative to the outlet aperture by the first channel.

7. The nebuliser outlet of claim 6, wherein at least part of the first channel has a cross sectional area that is approximately equal to a cross sectional area of the liquid capillary.

8. The nebuliser outlet assembly of claim 1, wherein the nebuliser outlet assembly is configured such that when the one or more second parts are removed from the first part, at least an outlet end of the first channel is exposed.

9. The nebuliser outlet assembly of claim 1, wherein the nebuliser outlet assembly is configured such that when a capillary is received by the first channel and when the one or more second parts are removed from the first part, an outlet end of the liquid capillary is exposed.

10. The nebuliser outlet of claim 9, wherein the nebuliser outlet assembly is configured such that when a capillary is received by the first channel and when the one or more second parts are removed from the first part, a distance between an outlet of the liquid capillary and the outlet aperture and/or the outlet end in an axial direction that extends along a length of the nebuliser outlet assembly and/or along a length of the liquid capillary can be determined.

11. A nebuliser comprising:
the nebuliser outlet assembly of claim 1; and
a liquid capillary;
wherein the liquid capillary is retained by the first channel.

12. The nebuliser of claim 11, wherein the nebuliser is configured such that, when the liquid capillary is installed in the nebuliser outlet, the distance between an outlet end of the capillary and the outlet end of the nebuliser outlet is adjustable.

13. An ion source comprising the nebuliser outlet assembly or the nebuliser of claim 1.

14. The ion source of claim 13, wherein the ion source comprises an Electrospray Ionisation (ESI) ion source, a Desorption Electrospray Ionisation (DESI) ion source, a Desorption Electro-Flow Focusing Ionisation (DEFFI) ion source, an impactor ion source, or an Atmospheric Pressure Chemical Ionisation (APCI) ion source.

15. An analytical instrument comprising the nebuliser outlet assembly, the nebuliser, or the ion source of claim 1.

16. The analytical instrument of claim 15, wherein the analytical instrument comprises a mass and/or ion mobility spectrometer.

17. A method of nebulising a liquid, the method comprising using the nebuliser outlet assembly and/or the nebuliser of claim 1 to nebulise a liquid.

18. A method of ionisation, the method comprising using the nebuliser outlet assembly and/or the nebuliser and/or the ion source of claim 1 to ionise an analyte.

19. A method of assembling a nebuliser outlet assembly that comprises:
a first part and one or more second parts that include an inlet end and an outlet end, and a first channel arranged between the inlet end and the outlet end, wherein the first channel is configured to receive a capillary;
the method comprising:
inserting a liquid capillary into the first channel when the one or more second parts are removed from the first part such that at least some of the first channel is exposed; and then
attaching the one or more second parts to the first part so as to form the first channel.

20. The method of claim 19, wherein inserting the liquid capillary into the first channel comprises inserting the liquid capillary into the first channel such that a distance between an outlet of the liquid capillary and the outlet aperture and/or the outlet end in an axial direction that extends along a length of the nebuliser outlet assembly and/or along a length of the liquid capillary is set to a desired distance.
